(19) United States
(12) Patent Application Publication
(10) Pub. No.: US 2008/0319184 A1
(43) Pub. Date: Dec. 25, 2008

(54) Uses of Dinucleotide Polyphosphate Derivatives

(76) Inventors: Andrew David Miller, London (GB); Michael Wright, London (GB); Julian Alexander Tanner, Hong Kong (HK); Natalya Lozovaya, London (GB)

Correspondence Address: SUZANNAH K. SUNDBY;SMITH, GAMBRELL & RUSSEL, LLP, 1130 Connecticut Avenue, NW, Suite 1130, WASHINGTON, DC, 20036, US

(21) Appl. No.: 11/883,661

(22) PCT Filed: Feb. 1, 2006
(86) PCT No.: PCT/GB06/00343

§ 371 Date: Aug. 27, 2008

(30) Foreign Application Priority Data
Feb. 3, 2005 (GB) 0502250.4

(51) Int. Cl. C07H 21/02

(52) U.S. Cl. 536/26.26

(57) ABSTRACT

The present invention provides the use of analogues and derivatives of dinucleoside polyphosphates with formula (I) or a pharmaceutically acceptable salt thereof, in the manufacture of a medicament for use in one or more of: the treatment of ischemia, inducing ischemic tolerance, modulating cerebral ischemia, to delay the onset of a hypoxic depolarisation stage when ischemic events are initiated; as a neurological protection agent; as a tissue protection agent; the treatment of pain; and the treatment of inflammation, wherein X, is selected from wherein X¹ and X² are independently selected from H, Cl, Br and F; each Y is independently selected from S and O; each Z is independently selected from —CX³X⁴—,—NH—,—O—; wherein X³ and X⁴ are selected from H, Cl, Br and F; B1 and B2 are independently selected from adenine, guanine, xanthine, thymine, uracil, cytosine and inosine; S₁ and S₂ are independently selected from ribose, open chain ribose, 2′-deoxyribose, 3′deoxyribose and arabinofuranoside. V is selected from 0, 1, 2, 3, 4 and 5; W is selected from 0, 1, 2, 3, 4 and 5; and V plus W is an integer from 2 to 6.

The present invention relates to the use of analogues and derivatives of dinucleoside polyphosphates.

Dinucleoside polyphosphates are a group of compounds comprising two nucleoside moieties linked by a polyphosphate bridge. Dinucleoside polyphosphates form an important family of compounds and are thought to have both intracellular and extracellular biological roles.^{1,2}

One dinucleoside polypeptide of particular interest is diadenosine 5′,5′″-P¹,P⁴-tetraphosphate (Ap₄A). Ap₄A is thought to function in cellular responses to cell proliferation and environmental stresses in prokaryotes and lower eukaryotes, as well as to play a role in extracelluar signalling in higher eurkaryotes.^{3,4} It has also been reported that Ap₄A may have protective effects in the cortex and midbrain in defined rat models of stroke and Parkinson's disease.⁵

A number of synthetic methods have been reported for the preparation of dinucleoside polyphosphates and attempts have been made to study their biological roles in more detail.^{6,7} Despite this, and the fact that many of these compounds are widely found in nature and have been known for a number of years,³ it has proved difficult to define the biological functions of such compounds. Indeed, the confusion over the role of such compounds has led to an ambiguous suggestion that they could act as either “friend or foe”.⁸ In general there have been considerable difficulties in obtaining results that are sufficiently reproducible to correlate biological functions in vivo or ex vivo with the presence of dinucleoside polyphosphates, reasons for this are not clear but could be related to hydrolytic lability.³

Attempts to study and use dinucledside polypeptides have also been hampered by the frequent difficulties encountered in isolating and purifying such compounds from natural sources. For example, diadenosine polyphosphates (ApₙA; n=2-6) appear to be highly unstable to specific enzymatic and non-specific hydrolysis in biological fluids and tissue samples.^{3,10}

The present invention alleviates the problems of the prior art.

In one aspect the present invention provides the use of a compound of formula (1):

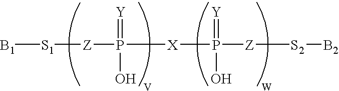

or a pharmaceutically acceptable salt thereof,

in the manufacture of a medicament for use in one or more of:

treatment of ischemia,

as a neurological protection agent;

as a tissue protection agent;

treatment of pain; and

treatment of inflammation;

wherein X, is selected from

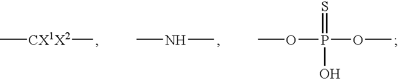

wherein X¹ and X² are independently selected from H, Cl, Br and F;

each Y is independently selected from S and O;

each Z is independently selected from

—CX³X⁴—, —NH—, —O—;

wherein X³ and X⁴ are selected from H, Cl, Br and F;

B₁ and B₂ are independently selected from adenine, guanine, xanthine, thymine, uracil, cytosine and inosine;

S₁ and S₂ are independently selected from ribose, 2′-deoxyribose, 3′deoxyribose, arabinofuranoside and ring opened forms thereof.

V is selected from 0, 1, 2, 3, 4 and 5;

W is selected from 0, 1, 2, 3, 4 and 5; and

V plus W is an integer from 2 to 6.

We have found that by our choice of X and Z groups we provide use of compounds (and novel compounds) which give persistent and reproducible biological outcomes in the presence of biological fluids and tissue samples. Thus a use and novel compounds are provided which allow for reproducible effects in vivo.

For ease of reference, these and further aspects of the present invention are now discussed under appropriate section headings. However, the teachings under each section are not necessarily limited to each particular section.

### PREFERRED ASPECTS

X

X is selected from

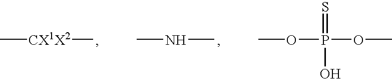

wherein X¹ and X² are independently selected from H, Cl, Br and F.

In one aspect X is —NH—.

In one aspect X is

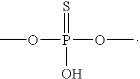

In one aspect X is —CX¹X²—.

In one aspect at least one of X¹ and X² is H.

In one aspect at least one of X¹ and X² is Cl.

In one aspect at least one of X¹ and X² is Br.

In one aspect at least one of X¹ and X² is F.

Preferably both X¹ and X² are H.

Preferably X is —CX¹X²— and X¹ and X² are both H.

Y

Each Y is independently selected from S and O;

In one aspect at least one Y is S.

In one aspect each Y group is S.

In one aspect at least one Y is O.

Preferably each Y group is O.

Z

Each Z is independently selected from

—CX³X⁴—, —NH—, —O—

wherein X³ and X⁴ are selected from H, Cl, Br and F;

In one aspect at least one Z is —CX³X⁴—

In one aspect each Z is —CX³X⁴—.

In one aspect at least one of X³ and X⁴ is H.

In one aspect at least one of X³ and X⁴ is Cl.

In one aspect at least one of X³ and X⁴ is Br.

In one aspect at least one of X³ and X⁴ is F.

Preferably both X³ and X⁴ are H.

Preferably Z is —CX³X⁴— and X³ and X⁴ are both H.

In one aspect at least one Z is —NH—.

In one aspect each Z is —NH—.

In one aspect at least one Z is —O—.

Preferably each Z is —O—.

B₁ and B₂

B₁ and B₂ are independently selected from adenine, guanine, xanthine, thymine, uracil, cytosine and inosine;

In one aspect at least one of B₁ and B₂ is uracil.

In one aspect at least one of B₁ and B₂ is guanine.

Preferably at least one of B₁ and B₂ is adenine.

Preferably at least one of B₁ and B₂ is adenine and the other of B₁ and B₂ is guanine.

Preferably at least one of B₁ and B₂ is adenine and the other of B₁ and B₂ is uracil.

Preferably B₁ and B₂ are both adenine.

S₁ and S₂

S₁ and S₂ are independently selected from ribose, 2′-deoxyribose, 3′deoxyribose, arabinofuranoside and ring opened forms thereof.

Preferably at least one of S₁ and S₂ is ribose.

Preferably at least one of S₁ and S₂ is a ring opened form of ribose.

Preferably at least one of S₁ and S₂ is ribose and the other of S₁ and S₂ is a ring opened form of ribose.

Preferably S₁ and S₂ are the same.

Preferably S₁ and S₂ are ribose.

V and W

V is selected from 0, 1, 2, 3, 4 and 5.

W is selected from 0, 1, 2, 3, 4 and 5.

V plus W is an integer from 2 to 6, that is the sum of V and W may be 2, 3, 4, 5 or 6.

Preferably V is 2.

Preferably W is 2.

Preferably V plus W is 4.

### Other Aspects and Features

Preferably, the compound of formula (1) is:

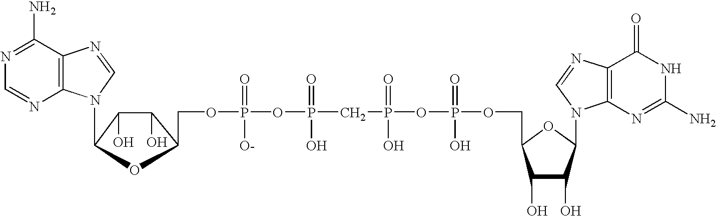

Preferably the compound of formula (1) is:

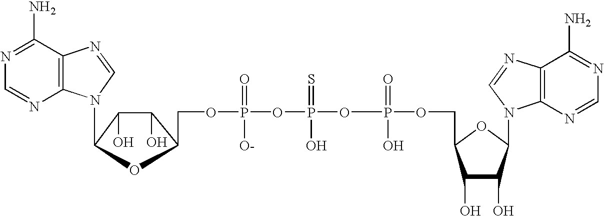

Preferably the compound of formula (1) is:

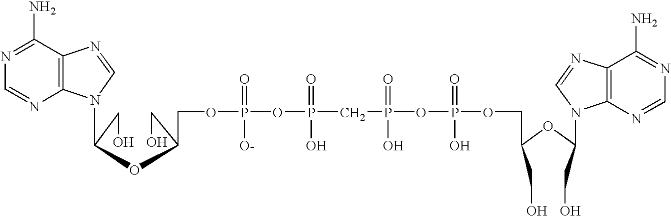

Preferably the compound of formula (1) is:

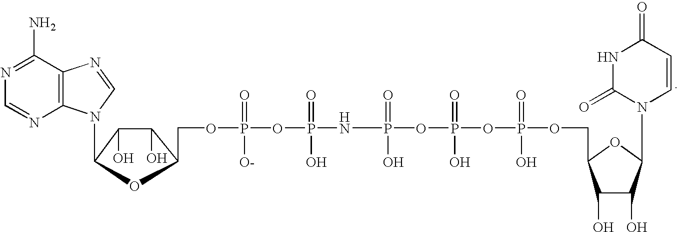

In a further aspect the present invention provides the use of compound of formula (1) as described herein, or a pharmaceutically acceptable salt thereof in the manufacture of a medicament for use in one or more of:

(a) treatment of diseases and medical conditions associated with P2-receptors;

(b) treatment of diseases and medical conditions associated with A1 adenosine receptors;

(c) moderating the activity of P2-receptors;

(d) moderating the activity of A1 adenosine receptors; and

(e) for modulating K+ influx via G protein-gated inwardly rectifying K⁺ (GIRK) channels in mammalian cells.

In a further aspect, the present invention provides a compound selected from:

(a) AppₛpA

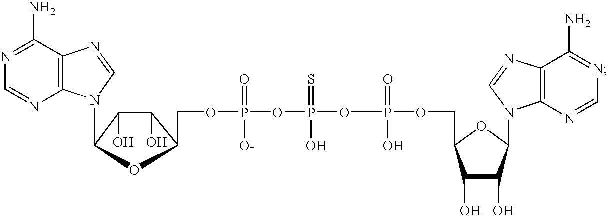

(b) A_{diol}ppCH2ppA_{diol}

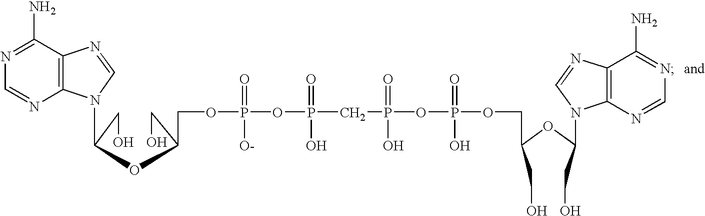

(c) AppNHpppU

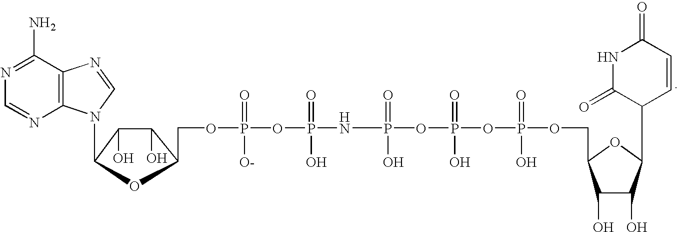

Preferably the compound is AppₛpA.

Preferably the compound is A_{diol}ppCH2ppA_{diol}.

Preferably the compound is AppNHpppU.

Ischemia

In one aspect, the present invention relates to the treatment of ischemia and ischemic related diseases and disorders. These treatments may include inducing ischemic tolerance, modulating cerebral ischemia and delaying the onset of a hypoxic depolarisation stage when ischemic events are initiated. Ischemic conditions occur when there is an inadequate supply of blood to an organ or a part of a human or animal body. As a consequence of this inadequate supply of blood, the organ or part of the body is deprived of oxygen and nutrients, such as glucose. This can result in the organ or part of the body being damaged. For example, if the blood supply to any portion of the central nervous system (CNS) is interrupted, the nerve cells (or neurons) of that portion of the CNS will rapidly degenerate.

In particular, the present invention may relate to the use of compounds in the manufacture of a medicament for the treatment of the following disorders: focal ischemia; global ischemia; cerebral ischemia; neuronal cell ischemia, such as the neuronal cell ischemia associated with spinal injuries and head trauma; myocardial ischemia; cardiovascular diseases, selected from the group: hypertension, angina, stable and unstable angina, Prinzmetal angina, arrhythmia, thrombosis, embolism, and congestive heart failure including chronic or acute congestive heart failure; or a disease characterised by ischemia of lower legs due to peripheral vascular disease, including intermittent claudication; a disease characterised by spasms of smooth muscle, selected from the group: spasms of the ureter, spasms of the bladder, uterine cramps, and irritable bowel syndrome; or in the prevention of vasoconstriction and/or ischemic tissue damage during a surgical procedure, selected from the group: bypass grafts, angiography, angioplasty, organ preservation during transplant, hypertensive crisis or post operative hypertension.

Neurological Diseases and Disorders

The present invention may be useful in the treatment of neurological diseases and disorders, in particular, in the treatment of neuronal cells. Such treatments include the treatment of brain trauma, brain or cerebrovascular ischemia, neurodegenerative diseases, poisoning of neuronal cells, and the preservation of neuronal grafts.

Neurodegenerative diseases are a group of disorders characterised by changes in the normal neuronal function, which may lead to neuronal death (most of these diseases are associated, especially in the later stages, with severe neuronal loss). These neurodegenerative diseases may include amyotrophic lateral sclerosis, Alzheimer's disease, Parkinson's disease and Huntington's disease.

Pain

In another aspect, the present invention may be useful in the treatment of pain. Such treatments may include the treatment of the pain associated with joint conditions (such as rheumatoid arthritis and osteoarthritis), pain associated with cancer, post-operative pain, postpartum pain, the pain associated with dental conditions (such as dental caries and gingivitis), the pain associated with bums (including sunburn), the treatment of bone disorders (such as osteoporosis, hypercalcaemia of malignancy and Paget's disease), the pain associated with sports injuries and sprains.

Inflammation

In another aspect, the present invention may relate to the treatment of inflammation. Inflammation may be caused by a variety of conditions, so for example, the present invention may relate to the treatment of arthritis, myocarditis, encephalitis, transplant rejection, systemic lupus erythematosis, gout, dermatitis, inflammatory bowel disease, hepatitis, or thyroiditis.

Stress

In another aspect, the present invention may relate to the treatment of chemical and/or environmental stress. In particular, the present invention may relate to the use of compounds to induce neurological preconditioning. Following administration of suitable compounds, such neurological preconditioning enables the neurological tissue to tolerate and/or survive levels of chemical and/or environmental stress which would normally prove lethal. This use of compounds described in the present invention may relate to of these compounds to elicit nitric oxide (NO), which can act as a mediator in the preconditioning of tissues to chemical and/or environmental stress.

The present invention will now be described in further detail by way of example only with reference to the accompanying figures in which:

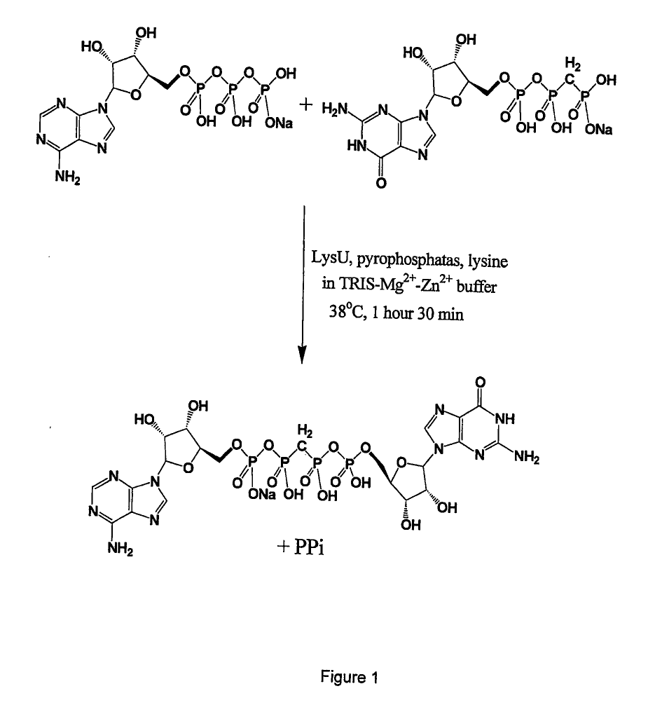

FIG. 1 shows the synthesis of AppCH₂ppG;

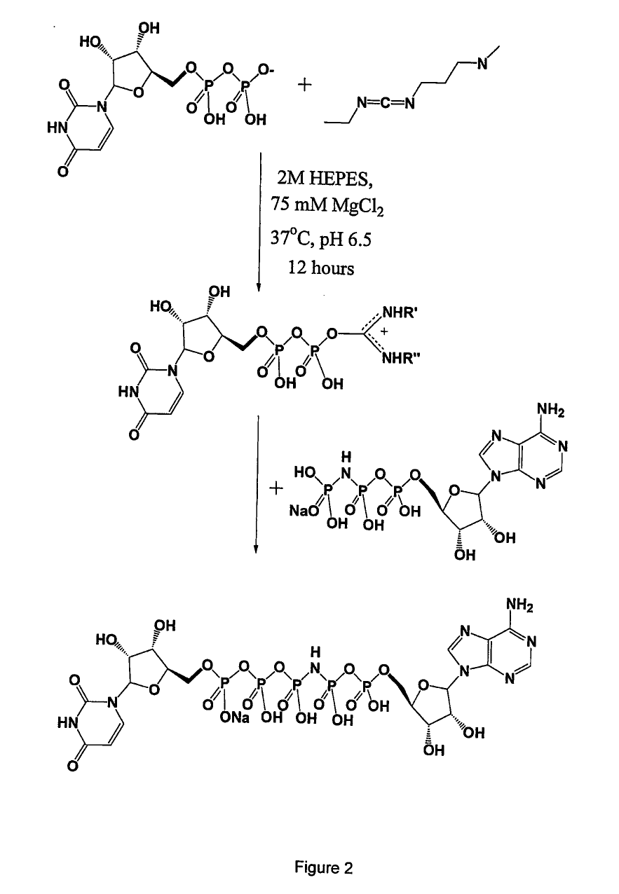

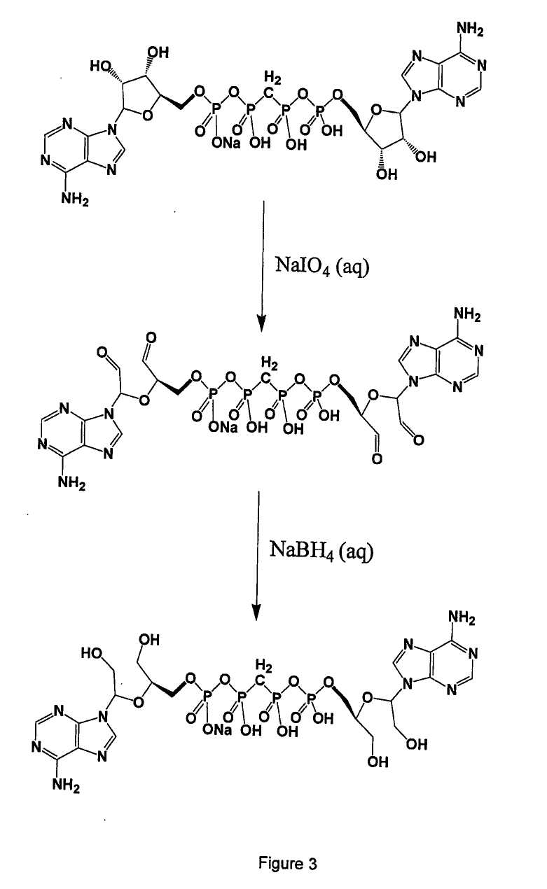

FIG. 2 shows the synthesis of AppNHpppU;

FIG. 3 shows the synthesis of A_{diol}ppCH₂ppA_{diol}

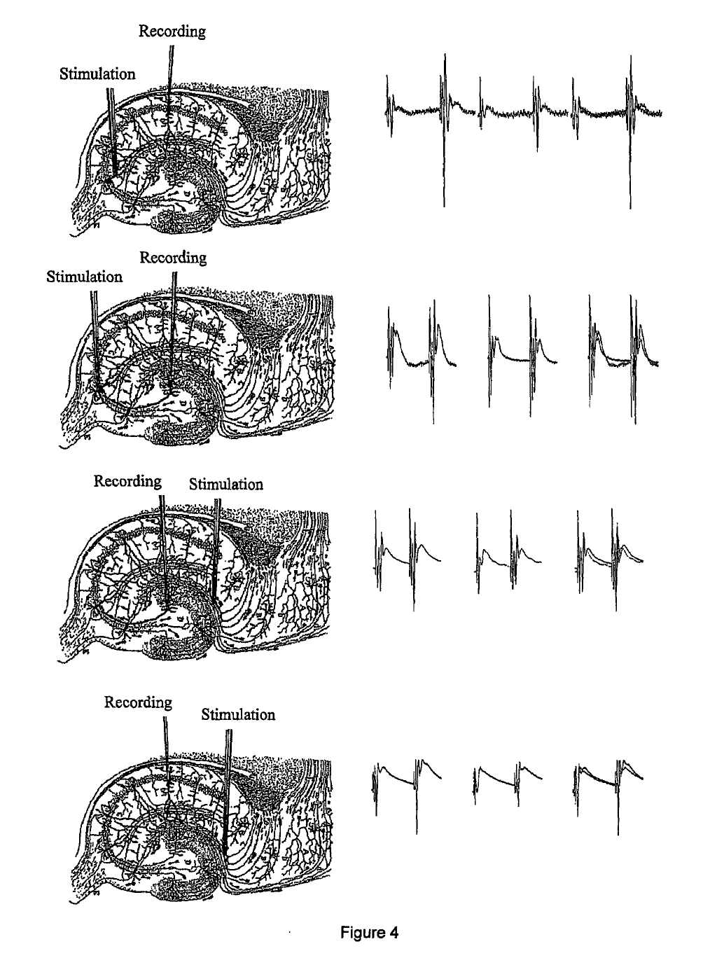

FIG. 4 shows a summary diagram of orthodromically (top 2) and antidromically (bottom 2) induced population spikes, illustrating electrode positions;

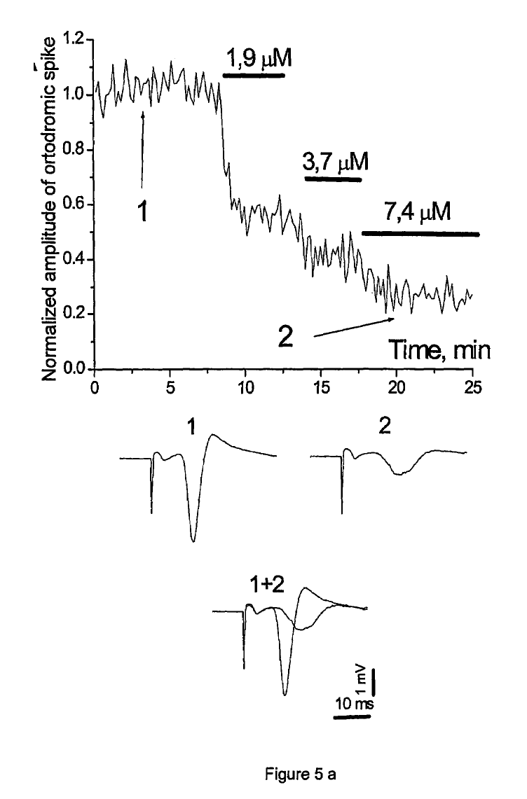

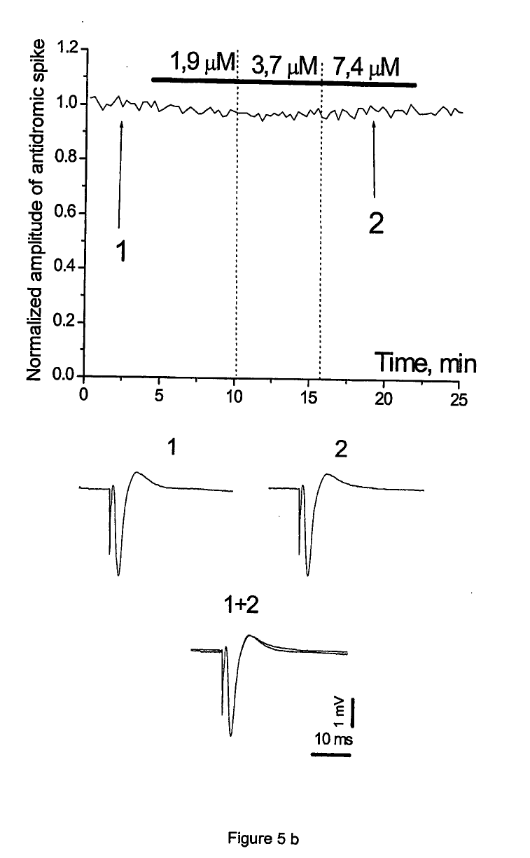

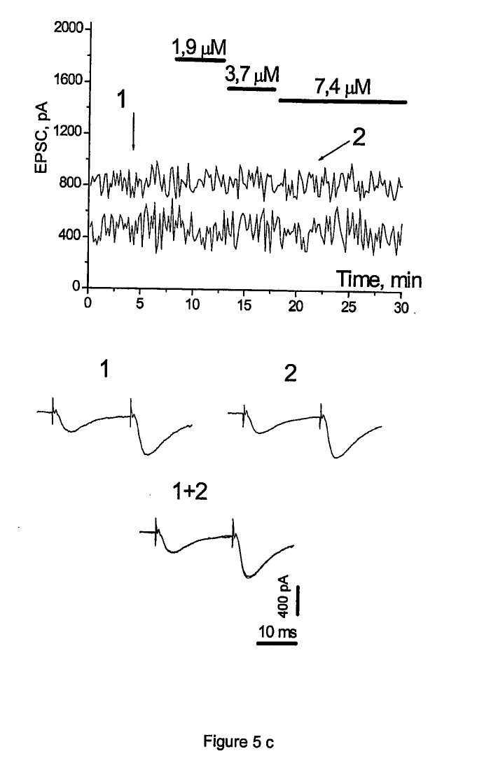

FIG. 5 shows the effect of increasing amounts of AppCH₂ppA on orthodromically induced population spikes (FIG. 5A), antidromically induced population spikes (FIG. 5B) and excitatory postsynaptic currents, EPSCs, (FIG. 5C);

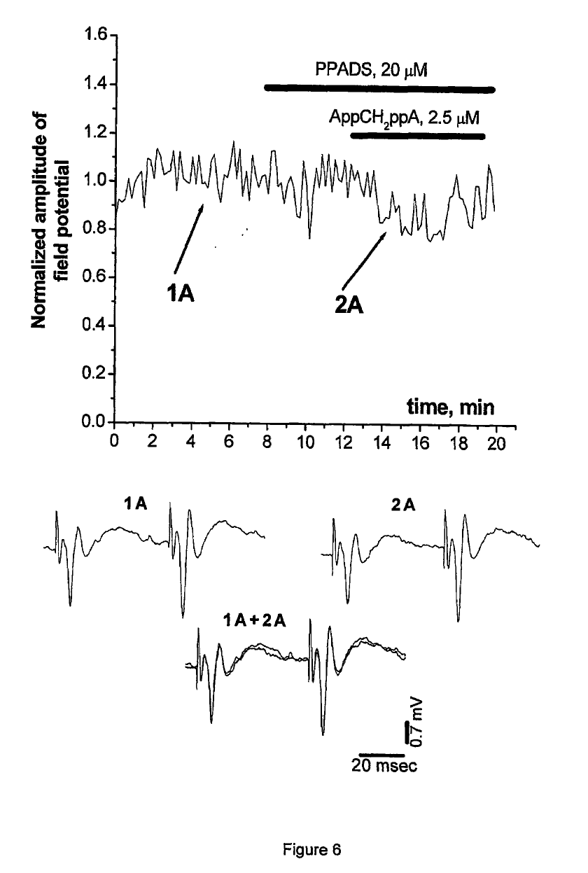

FIG. 6 shows the influence of pyridoxal-phosphate-6-azophenyl-2′,4′-disulphonic acid (PPADS) and AppCH₂ppA on orthodromic spikes;

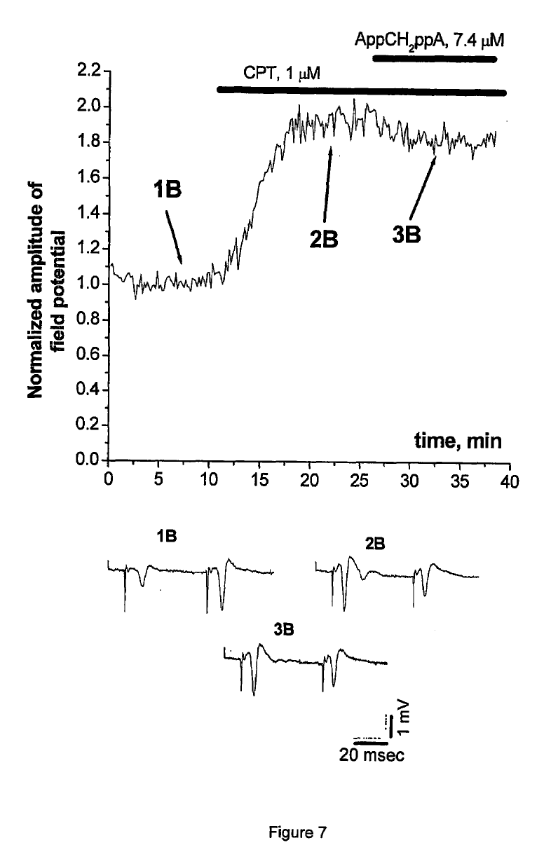

FIG. 7 shows the influence of cyclopentyl teophylline (CPT) and AppCH₂ppA on orthodromic spikes;

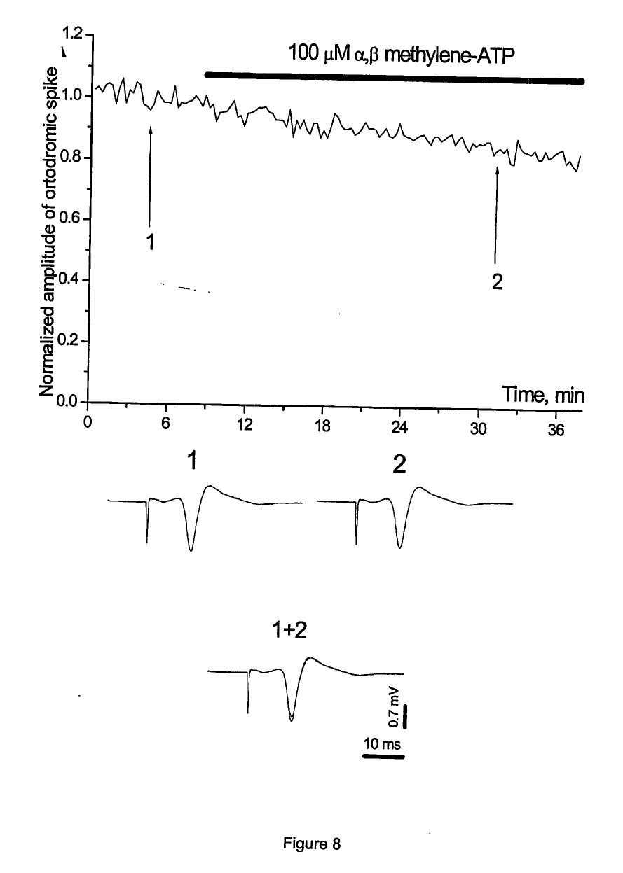

FIG. 8 shows the effect of α,β-methylene-ATP on orthodromic spikes;

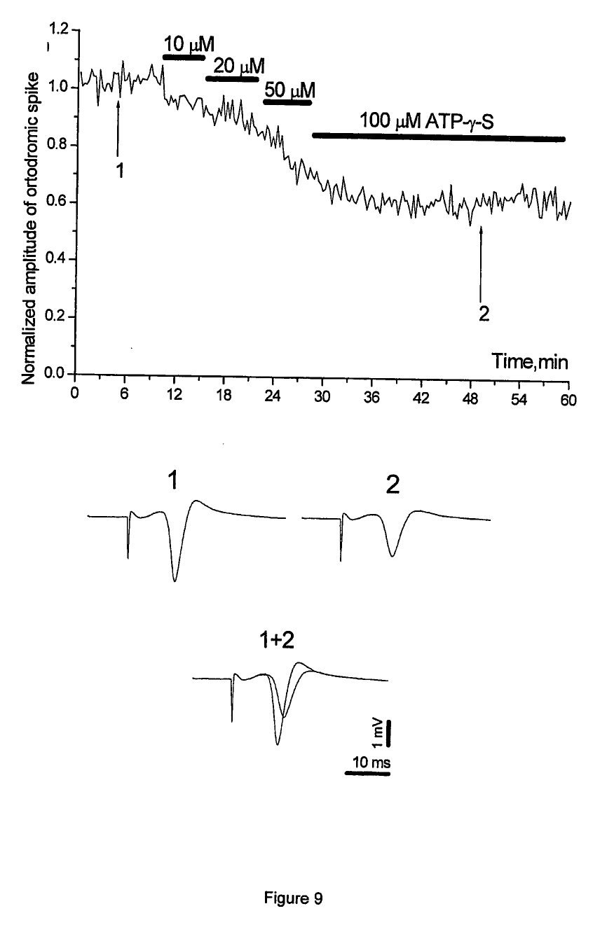

FIG. 9 shows the effect of increasing amounts of ATPγS on orthodromic spikes;

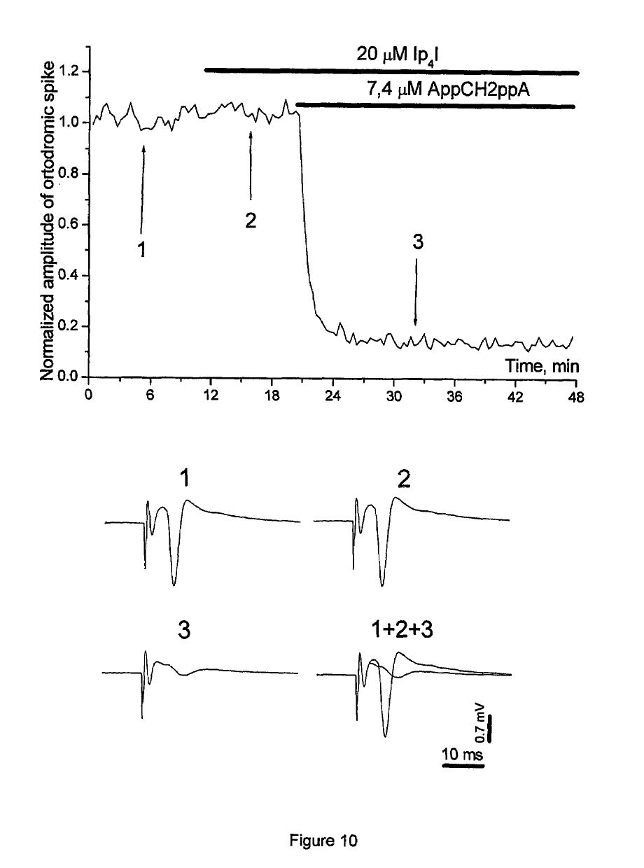

FIG. 10 shows the influence of diinosine tetrahydrophosphate (Ip₄I) and AppCH₂PpA on orthodromic spikes;

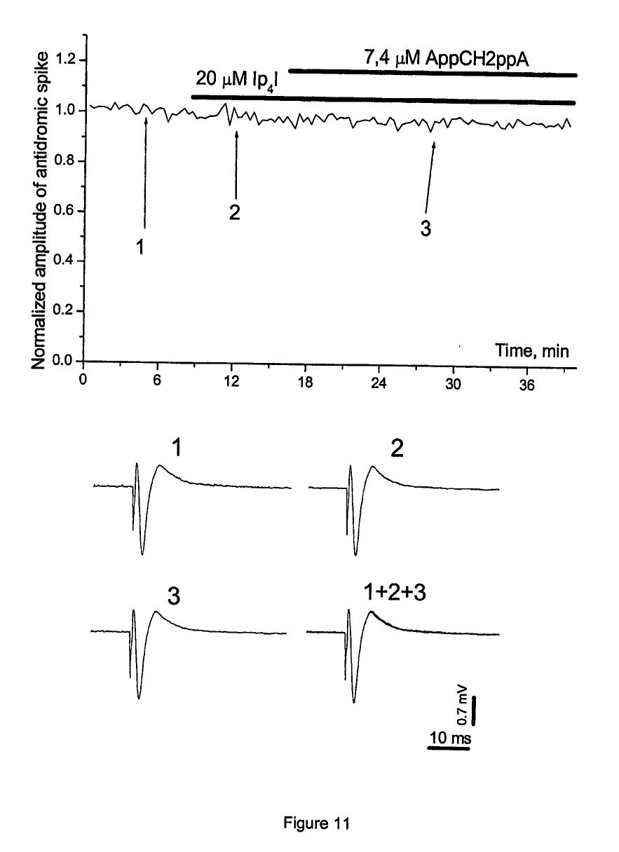

FIG. 11 shows the influence of diinosine tetrahydrophosphate (Ip₄I) and AppCH₂ppA on antidromic spikes;

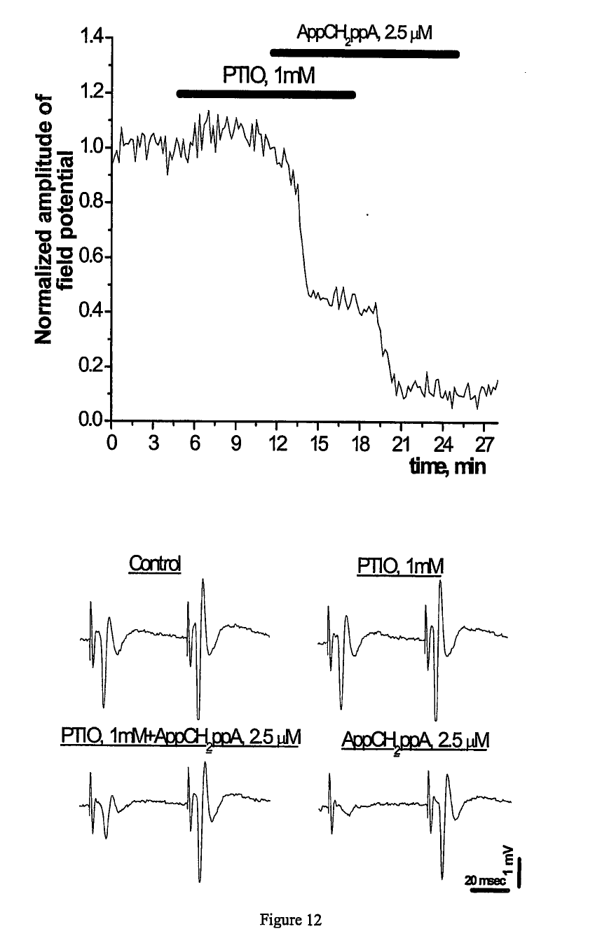

FIG. 12 shows the influence of 2-phenyl-4,4,5,5-tetramethylimidazoline-1-oxyl 3-oxide, (PTIO) and AppCH₂ppA on orthodromic spikes;

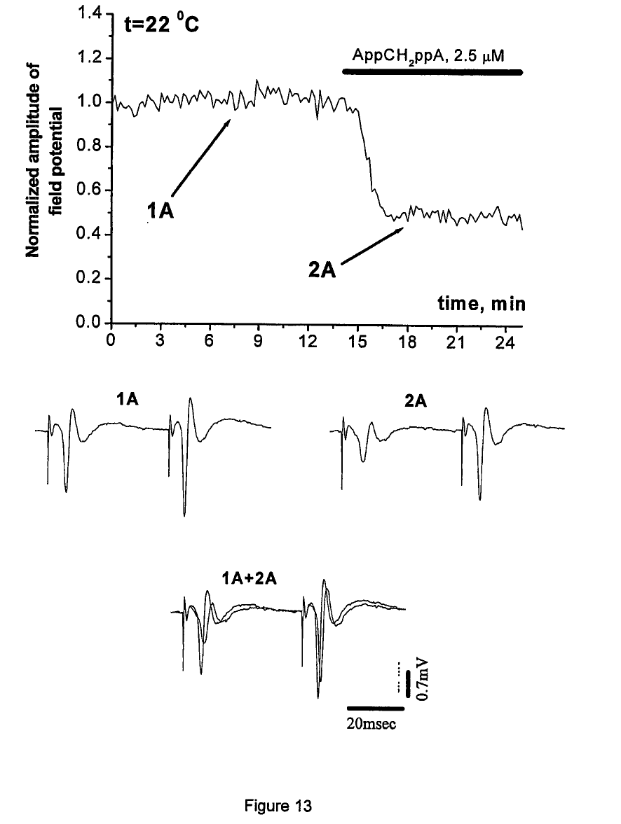

FIG. 13 shows the shows the influence of AppCH₂ppA on orthodromic spikes at 22° C.;

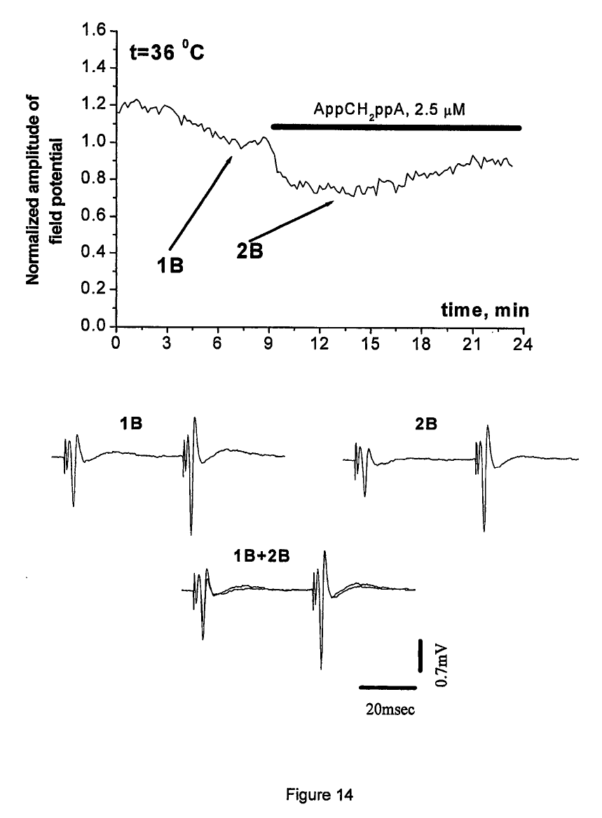

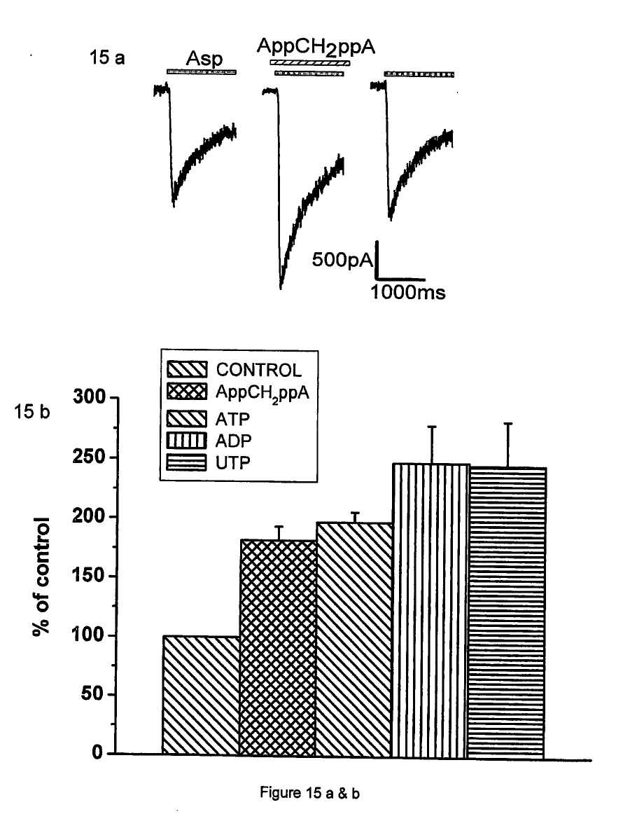

FIG. 14 shows the shows the influence of AppCH₂ppA on orthodromic spikes at 36° C.

The present invention will now be described in further detail in the following examples.

### EXAMPLES

Electrospray mass spectroscopy (ES-MS) carried on a Bruker Esquire 3000 machine set to 100% fragment strength. Samples were applied in 1:1 acetonitrile:water containing 0.1% acetic acid. Proton and phosphorous NMR spectra were recorded on a 400 MHz Bruker Ultrashield, with samples in D₂O at 300K. 64 scans were used for proton spectra, 1024 scans for phosphorous. For simplicity, only those ¹H NMR signals particularly useful for compound identification have been described.

### Preparation of Compounds

AppCH₂ppG

10×1 ml portions of LysU reaction mixture was made up in aliquots. This mixture contained 2 mM L-lysine, 10 mM MgCl₂, 160 μM ZnCl₂, and 6U of pyrophosphatase in 50 mM Tris-HCl buffer, pH 8.0.^{7,11} Nucleotides were added to 8 mM ATP and 4 mM GMPPCP (α,β-methylene-guanosine 5′-triphosphate), the mixtures were vortexed and LysU added to 9 μM concentration (dimer). The mixes were then incubated at 38° C. and the reaction monitored by HPLC using a 2 ml SOURCE 15Q (Amersham Biosciences) ion exchange column, packed in 50 mM Tris-HCl buffer (pH 8.0) and eluted with a 0-0.5 M gradient of salt over 5 mins at 2 ml/min.⁹ After 25 minutes the ATP and GMPPCP peaks (previously seen at 5.5 min and 5.2 min) were lost and had been replaced with an Ap₄A peak at 7.2 min and the target at 6.8 min. Continued incubation converted the Ap₄A to Ap₃A (5.1 min) after 1 hour, without degradation of the target. AppCH₂ppG was purified using a 60 ml SOURCE Q column packed in water, eluted with a 0-2M gradient of TEAB (triethylammonium hydrogencarbonate buffer) over 30 mins at 8 ml/min, and lyophilised for storage at −20° C. ^{9,12} SoQ/NaCl HPLC showed a single peak and the product has an ES-MS (M-H) of 848.9 m/z. ¹H NMR: 8.39 (1H, s, 8-H-AD), 8.15 (1H, s, 2-H-Ad), 8.03 (1H, s, 8-H-Gu), 6.04 (1H, s, 1′-H-rib[Ad]), 5.82 (1H, s, 1′-H-rib[Gu]), 2.46 (2H, t, O—CH₂—O), 2-H-Gu not seen as presumably labile, ³¹P NMR: 8.74 (2P, m, β-P), −10.63 (2P, m, α-P). Yield was 90%+

AppNHpppU

LysU synthesis of AppNHpppU would require NH substituted adenosine tetraphosphate, which is not available. Therefore, we used a chemical coupling based on the dehydration agent EDC (1-ethyl-3-(3-dimethylaminopropyl)carbo-diimide).¹³ AMPPNHP (adenosine 5′-(β,γ-imido) triphosphate, 50 mg) and UDP (uridine diphosphate, 150 mg) were dissolved 2 M HEPES pH 6.5 with 75 mM MgCl₂ in 10×1 ml aliquots. 400 mg of EDC was added to each aliquot, and the mixtures incubated at 37° C., again monitoring by SoQ/NaCl HPLC (UDP 3.8 min, AMPPNHP 4.7 min). After 12 h, two product peaks were seen at 6.1 min and 7.0 min. They were extracted with the SoQ/TEAB column and lyophilised. ^{9,12} Both products showed a single band by SoQ/NaCl HPLC, the 6.1 min had an ES-MS (M-H) of 788.6 m/z and was identified as Up₄U, whilst the 7.0 min had 890.7 m/z which matches ApppNHppU. ApppNHppU ¹H NMR: 8.52 (1H, s, 8-H-Ad), 8.21 (1H, s, 2-H-Ad), 7.87 (1H, s, 6-H-Ur), 6.08 (1H, s, 1′-H-rib[Ad]), 5.90 (2H, d 1′-H-rib[Ur] and 5-H-Ur), 3-H-Ur not seen (presumably labile), O—NH—O obscured, ³¹p NMR: three close bands seen of complex multiplicity, −10.89, −10.97 and −11.25. The latter is tentatively identified as an overlay of α-P, β-P and ε-P. Up₄U ¹H NMR: 7.91 (2H, s, 6-H-Ur), 5.93 (4H, s, 1′-H-rib and 5-H-Ur) ³¹P NMR: a single band (d m) is seen at −11.46 ppm. Yield was 45% with respect to initial AMPPNHP (though some starting material was recovered).

A_{diol}ppCH₂ppA_{diol}

AppCH₂ppA (100 mg, previously made by LysU coupling from ATP and AMPPCP [α,β-methylene-adenosine 5′-triphosphate])⁹ was dissolved in 2 ml distilled water. 150 μl of 0.3 M aqueous sodium periodate was added, followed after 10 mins, with 50 μl of 0.5 M aqueous sodium borohydride (warning: H₂ evolved). The reactions were monitored by SoQ/NaCl HPLC as normal, with the 5.8 min peak of AppCH₂ppA up-shifting to 6.2 min on oxidation to the dialdehyde and falling to 4.4 min on reduction to the diol. Separation by SoQ/TEAB ^{9,12} gave two main bands, which were extracted and lyophilised. Both showed single peaks by SoQ/NaCl HPLC with the greater product having a ES-MS (M-H) of 836.9 m/z and the lesser 585.7 m/z. These match A_{diol}ppCH₂ppA_{diol} and A_{diol}ppCH₂pp respectively. A_{diol}ppCH₂ppA_{diol} ¹H NMR: 8.34 (2H, s, 8-H-Ad), 8.13 (2H, s, 2-H-Ad), 5.96 (2H, s, 1′-H-rib), 3.97 (4H, s, 2′-H-rib), 3.80 (4H, s, 3′-H-rib), 3.70 (4H, s, rib-CH₂—O), 2.31 (2H, t, O—CH₂—O) ³¹P NMR: 7.17 (2P, q, β-P), −11.16 (2P, d, α-P). A_{diol}ppCH₂pp ¹H NMR: 8.41 (1H, s, 8-H-Ad), 8.22 (1H, s, 2-H-Ad), 6.01 (1H, s, 1′-H-rib), 4.01 (2H, s, 2′-H-rib), 3.74 (4H, m, 3′-H-rib and rib-CH₂—O), 2.36 (2H, t, O—CH₂—O) ³¹P NMR: 7.50 (1P, m, β-P), 6.45 (1P, q d, γ-P), −10.56 (1P, d, δ-P), −11.21 (1P, d m, α-P). Yield was 80%.

Biological Tests

The following biological data was acquired in order to determine the effects and mechanism of dinucleoside polyphosphate analogues. Critically, where compounds are shown to be P2 and/or A1 receptor agonists, then they would be expected to possess an impressive range of potential therapeutic properties. In the past 25 years there have been many studies suggesting that P2 and/or A1 receptor agonists used in both the central nervous system (CNS) and peripheral nervous system (PNS) will facilitate or synergise with the actions of a wide variety of CNS active drugs that include analgesics, antipsychotics, antidepressants, anxiolytics, nootropics/cognition enhancers and the various agents effective in stroke related CNS damage. Furthermore, such receptor agonists also appear to be potent neurological compounds in their own right.

Against Stroke and Ischaemia

Chemical agents acting as A1 receptor agonists appear to promote stable neuronal membrane potentials that result in the inhibition of neuronal excitability and excitatory amino acid (EAA) release.²² Blockade of EAA release thus prevents the neurotoxic sequelae associated with activation of N-methyl-D-aspartate (NMDA) receptor. A1 receptor agonists can also reduce stroke related cell death and hippocampal neurodegeneration.²²

Against Epilepsy

Chemical agents acting as A1 receptor agonists reduce epileptic seizure activity induced by a variety of chemical and electrical stimuli in animal models.^{23,24} In electrically kindled seizure models, A1 receptor agonists are anticonvulsants that reduce seizure severity and duration without significantly altering seizure threshold.²³

Against Neurodegeneration

There is wide acceptance that neuronal hyper-excitability associated with ischaemia, hypoxia and epilepsy also underlies the neurodegenerative processes associated with aging. Chemical agents acting as P2 and/or A1 receptor agonists reduce EAA neurotoxicity and the resultant changes in calcium homeostasis that lead to nerve cell death may reflect an acute manifestation of more subtle, long term changes that are associated with Alzheimer's Disease (AD) and Parkinson's Disease (PD).²⁵ Agonists are potent anti-inflammatory agents²⁶ acting to inhibit free radical production and may thus provide additional benefit in providing potential AD treatments over and above direct effects on neurotransmitter-mediated neuronal events. A1 receptor agonists can reduce the high affinity state of striatal Dopamine (DA) D1 receptors.²⁷ Functionally, the A1 agonist blocks DA D1 receptor-mediated locomotor activation in reserpinized mice. Alternatively, agonists can attenuate peri-oral dyskinesias induced by selective DA D1 activation in rabbits. This dynamic inter-relationship between dopaminergic and purinergic systems in the neurochemistry of psychomotor function offers new possibilities for the amelioration of dopaminergic dysfunction via A1 receptor modulation.

Against Wakefulness

Direct administration of chemical agents acting as A1 agonists into the brain elicits an EEG profile similar to that seen in deep sleep, manifested as an increase in REM sleep with a reduction in REM sleep latency that results in an increase in total sleep.²⁸ A1 selective agonists may suppress slow wave sleep (SWS) and paradoxical sleep (PS) prior to eliciting an increase in SWS.

Against Pain

The application of ATP to sensory afferents results in hyper-excitability and the perception of intense pain. The nucleotide can also induce nociceptive responses at local sites of administration and can facilitate nociceptive responses to other noxious stimuli.²⁹ The pronociceptive actions of ATP are mediated via P2X receptors present on sensory afferents and in the spinal cord. Homomeric P2X3 and heteromeric P2X2/3 receptors are highly localized on the sensory nerves that specifically transmit nociceptive signals.³⁰ ATP is released from a number of cell types (e.g. sympathetic nerves, endothelial cells, visceral smooth muscle) in response to trauma³¹ and there is a substantive body of evidence that activation of P2X3 receptors may initiate and contribute to the peripheral and central sensitization associated with visceral nociception.³¹ P2X3 receptor expression is up-regulated in sensory afferents and spinal cord following damage to peripheral sensory fibers.³² Thus the development of selective, bio-available P2X3 receptor antagonists may be anticipated to provide novel compounds for the treatment of pain.

In contrast, the administration of chemical agents acting as A1 receptor agonists provides pain relief in a broad spectrum of animal models (e.g., mouse hot plate test, mouse-tail flick assay, rat formalin test, mouse abdominal constriction assay.^{29,33,34,35} A1 agonists are also effective in relieving neuropathic pain in rat models,³⁶ and inhibit pain-associated behaviour elicited by spinal injection of substance P and the glutamate agonist, NMDA. In mechanistic terms, A1 receptor agonists are known to inhibit the release of glutamate into the spinal fluid and also reduce cerebrospinal fluid levels of substance P in rat.^{29,37,38} Glutamate is a key mediator of the abnormal hyper-excitability of spinal cord dorsal horn neurons (central sensitization) that is associated with states of clinical pain.³⁹ Substance P is another key mediator of nociceptive responses.^{29,37,38} A1 agonists have also shown utility in relieving human pain.³⁸ Spinal administration of A1 agonist relieves allodynia in a neuropathic pain patients without affecting normal sensory perception. Infusion improves pain symptoms in clinical pain models reducing spontaneous pain, ongoing hyperalgesia and allodynia in patients with neuropathic pain. In addition, low dose infusions of agonists during surgery may reduce the requirement for volatile anesthetic and for post-operative opioid analgesia.^{37,40}

### General Methods

Hippocampal Slice Preparation

This study was carried out on 21-day old Wistar rats (WAG/GSto, Moscow, Russia). After rapid decapitation, rat brains were immediately transferred to a Petri dish with chilled (4° C.) solution of the composition 120 mM NaCl, 5 mM KCl, 26 mM NaHCO₃, 2 mM MgCl₂ and 20 mM glucose. Calcium salts were omitted to reduce possible neuronal damage. The solution was constantly bubbled with 95%O₂/5%CO₂ gas mixture to maintain pH7.4. Hippocampal slices (300-400 μM thick) were cut manually with a razor blade along the alveolar fibres to preserve the lamellar structure of excitatory connections. During preincubation and experiments, the slices were kept fully submerged in the extracellular solution, pH7.4, comprised of 135 mM NaCl, 5 mM KCl, 26 mM NaHCO₃, 1.5 mM CaCl₂, 1.5 mM MgCl₂, and 20 mM glucose, subjected to continuous bubbling with 95% O₂/5% CO) at 30-31° C. 25-50 mM picrotoxin (RBI, Natick, Mass., USA) was also included into the extracellular solution during experiments to suppress the inhibitory activity of interneurons. Electrophysiological measurements were recording after at least 2 h of preincubation.

Electrophysiological Measurements

Excitatory postsynaptic currents (EPSCs) were recorded by a standard whole-cell patch clamp technique in the CA1 subfield of the hippocampus in response to stimulation of the Schaffer collateral/commissural pathway. To prevent the spread of electrical activity from area CA3, mini-slices were prepared by making a cut orthogonal to the stratum pyramidale extending to the mossy fibre layer. The intracellular solution, pH 7.2, for patch pipettes was comprised of 100 mM CsF (Merck, Darmstadt, FRG), 40 mM NaH₂PO₄, 10 mM HEPES-CsOH, 10 mM Tris-HCl. 2-3 mM N-(2,6-dimethyl-phenylcarbamoylmethyl)-triethylammonium bromide (QX-314; Tocris Cookson, Bristol, UK) was routinely added to the intracellular solution to block voltage-gated sodium conductances. Patch pipettes were pulled from soft borosilicate glass on a two-stage horizontal puller. When fire-polished and filled with the intracellular solution, they had a resistance of 2-3MΩ. To visualise cell bodies of CA1 pyramidal neurones, the stratum oriens and alveus were removed with a saline jet from a micropipette. Currents were digitally sampled at 400 ms intervals by a 12-digit ADC board, filtered at 3 kHz, and data stored on a hard disk for further analysis. Access resistance was monitored throughout the experiments and ranged typically from 6 to 9MΩ. Data from cells where access resistance changed by more than 25% during the experiment were discarded. Extracellular field potentials were recorded using Ni/Cr electrodes. The population spikes were digitised and stored on computer disk. The effects of receptor agonists of antagonists were measured as the mean ratio I/Iₒ where I was the current under the substances action and Iₒ was the current in control saline. To stimulate the Schaffer collateral/commissural pathway input, a bipolar Ni/Cr electrode was positioned on the surface of the slice. Current pulses (10-100 pA) of 0.1-1 ms duration were delivered through the isolated stimulator HG 203 (Hi-Med, London, UK) at 0.066-0.2 Hz.

### Experiment 1

The time course of the changes of amplitude in orthodromically induced population spikes (FIG. 5A), antidromically induced population spikes (FIG. 5B) and excitatory postsynaptic currents, EPSCs, (FIG. 5C) in a CA1 zone of rat hippocampal slices prepared in accordance with the general procedure was measured. Over time, increasing amounts of Ap₂CH₂p₂A was applied to the rat hippocampal slice. Thus, 1.9 μm of Ap₂CH₂p₂A was applied after 10 mins, this was increased to 3.7 μm after 14 mins, and to 7.4 μm after 18 mins.

The electrode positions used to induce the orthodromically (top 2) and antidromically (bottom 2) induced population spikes in this experiment are illustrated in FIG. 4. AppCH₂ppA was found to produce reproducibly fast and reversible inhibition of orthodromically evoked field potentials in all synaptic pathways (FIG. 4) in hippocampus including CA3-CA1 synapses (FIG. 5A). To the right of the time course in FIG. 5A is shown the original traces of population spikes (five-fold averaged) corresponding with points 1 (control) and 2 (Ap₂CH₂p₂A effect) in the time course.

In contrast to the orthodromically evoked field potentials, the amplitudes of antidromic spikes (here and below CA3-CA1 synapses) (FIG. 5B) as well as EPSCs, recorded in CA1 pyramidal neuron, (FIG. 5C) remained unchanged. EPSC decay was also unchanged suggesting that AppCH₂ppA was not modulating the NMDA component of EPSCs either (FIG. 5C).

In contrast, the literature shows that Ap₄A induces the inhibition of excitatory postsynaptic currents as well as orthodromically evoked filed potentials.¹⁴ These results have also proven difficult to reproduce and are unreliable.

### Experiment 2

The time course of the changes of amplitude in orthodromically induced population spikes in CA1 zone of rat hippocampal slices prepared in accordance with the general procedure was measured. After 8 mins, pyridoxal-phosphate-6-azophenyl-2′,4′-disulphonic acid (PPADS) (20 μM) was applied. Then after 12 mins, Ap₂CH₂p₂A was applied.

It was found that using pyridoxal-phosphate6-azophenyl-2′,4′-disulphonic acid (PPADS) completely abolished the blocking effect of AppCH₂ppA on the orthodromic spikes (FIG. 6). FIG. 6 shows, on the left, the time course of the changes of amplitude in orthodromically induced population, and on the right it shows the original traces of population spikes (five-fold averaged) corresponding with points 1 (control) and 2 (Ap₂CH₂p2A/PPADS effect) in the time course. Since PPADS is a well-known broadband P2-receptor family antagonists^{15,16}, this result suggested that the observed AppCH₂ppA effects could be mediated by a novel P2-family receptor with unconventional pharmacology.

### Experiment 3

The time course of the changes of amplitude in orthodromically induced population spikes in CA1 zone of rat hippocampal slices prepared in accordance with the general procedure was measured. After 9 mins α,β-methylene-ATP (1001M) was applied (FIG. 8).

### Experiment 4

The time course of the changes of amplitude in orthodromically induced population spikes in CA1 zone of rat hippocampal slices prepared in accordance with the general procedure was measured. Over time, increasing amounts of ATPYS was applied to the rat hippocampal slice (FIG. 9). Thus, 10 μm of ATPYS was applied after 10 mins; this was increased to 20 μm after 15 mins; to 50 μm after 21 mins; and to 100 μm after 29 mins.

Experiments 3 and 4 are agonist experiments using known agonists of P2X and/or P2Y-family receptors. However, α,β-methylene-ATP (FIG. 8) and ATPγS (FIG. 9) were unable to inhibit orthodromically evoked field potentials in the same way as AppCH₂ppA.

The only effect observed was a weak, slowly developing inhibition at very high nucleotide concentrations (100 μM). Given the high concentrations of agonists used, such effects are, at best, non-specific. Therefore, it appears that AppCH₂ppA effects are unlikely to be mediated by the main P2X or P2Y-family receptors. This is perhaps surprising in view of the known capacity of ApₙAs to act as agonists of P2X₁₋₄, P2Y₁, P2Y₂ and P2Y₄ receptors in neurological tissue¹⁵.

### Experiment 5

The time course of the changes of amplitude in orthodromically induced population spikes in CA1 zone of rat hippocampal slices prepared in accordance with the general procedure was measured (FIG. 10. After 10 mins, diinosine tetraphosphate (Ip₄I) (20 μM) was applied. Then after 20 mins, Ap₂CH₂p₂A (7.4 μM) was applied.

### Experiment 6

The time course of the changes of amplitude in antidromically induced population spikes in CA1 zone of rat hippocampal slices prepared in accordance with the general procedure was measured (FIG. 11). After 10 mins, diinosine tetraphosphate (Ip₄I) (20 μM) was applied. Then after 16 mins, Ap₂CH₂p₂A (7.4 μM) was applied.

The possibility that the effects of AppCH₂ppA could be mediated by the P4-dinucleotide receptor previously identified¹⁷ on rat brain synaptic terminals was evaluated in Experiments 5 and 6. However, it was found that addition of even high concentrations of the P4 antagonist diinosine tetraphosphate (Ip₄I) (20 μM) did hot alter the effect of AppCH₂ppA on the changes of amplitude in orthodromically induced population spikes (FIG. 10). In addition, it was found that addition of Ip₄I (20 μM) did not cause AppCH₂ppA to have an effect on the changes of amplitude in antidromically induced population spikes (FIG. 11). These results completely rule out the possibility of P4-dinucleotide receptor mediation. Thus, these results suggest that a new P2-family receptor could be mediating the observed AppCH₂ppA effects.

### Experiment 7

The time course of the changes of amplitude in orthodromically induced population spikes in CA1 zone of rat hippocampal slices prepared in accordance with the general procedure was measured. After 10 mins, bicuculine (50 μM) was applied. Then after 20 mins, Ap₂CH₂p₂A (7.4 μM) was applied.

The AppCH₂ppA induced inhibition of orthodromically evoked field potentials was unaffected by the presence of bicuculine.

### Experiment 8

The time course of the changes of amplitude in orthodromically induced population spikes in CA1 zone of rat hippocampal slices prepared in accordance with the general procedure was measured. After 10 mins, hexamethonium (100 μM) was applied. Then after 20mins, Ap₂CH₂p₂A (7.4 μM) was applied.

The AppCH₂ppA induced inhibition of orthodromically evoked field potentials was unaffected by the presence of hexamethonium.

### Experiment 9

The time course of the changes of amplitude in orthodromically induced population spikes in CA1 zone of rat hippocampal slices prepared in accordance with the general procedure was measured (FIG. 7). After 10 mins, strychnine (500 nM) was applied. Then after 20 mins, Ap₂CH₂p₂A (7.4 μM) was applied.

The AppCH₂ppA induced inhibition of orthodromically evoked field potentials was unaffected by the presence of strychnine.

### Experiment 10

The time course of the changes of amplitude in orthodromically induced population spikes in CA1 zone of rat hippocampal slices prepared in accordance with the general procedure was measured. After 10 mins, cyclopentyl teophylline (CPT) (1 μM )was applied. Then after 26 mins, Ap₂CH₂p₂A (7.4 μM) was applied.

The AppCH₂ppA induced inhibition of orthodromically evoked field potentials was eliminated by the presence of CPT.

Experiments 7, 8, 9 and 10 investigate the location of the receptor that mediates the AppCH₂ppA effects. It can be deduced that the precise inhibition of orthodromic spikes must arise from the modulation of postsynaptic neuron excitability alone because AppCH₂ppA does not modulating synaptic transmissions at all. Furthermore as antidromically evoked field potentials are not modulated by AppCH₂ppA then the site of modulation must be located in postsynaptic CA1 dendrites. The dendrites of CA1 pyramidal neurons are well known as an important target for cortical modulation mediated via numerous receptors including cholinergic and GABAergic receptors. Accordingly, these experiments used known antagonists of γ-aminobutyric acid (GABA) (Experiment 7), muscarinic (Experiment 8) and glycine receptors (Experiment 9). However, none of these antagonists affected the AppCH₂ppA induced inhibition of orthodromically evoked field potentials. By contrast, the administration of CPT, an A1 adenosine receptor antagonist, was seen to eliminate the effect of AppCH₂ppA (FIG. 7), thereby suggesting that AppCH₂ppA effects are mediated instead by A1 adenosine receptor activation downstream of PPADS-sensitive P2 receptor activation.

This suggestion is supported by the fact that the administration of adenosine (5 μM) to hippocampal slices has been shown previously to inhibit orthodromic spikes¹⁸ in a similar fashion to AppCH₂ppA. However, adenosine administration was also shown to diminish EPSC amplitudes,¹⁹ in direct contrast with the effect observed following AppCH₂ppA administration (FIG. 5C). Therefore, AppCH₂ppA mediated effects are altogether more selective than adenosine alone.

### Experiment 11

The time course of the changes of amplitude in orthodromically induced population spikes in CA1 zone of rat hippocampal slices prepared in accordance with the general procedure was measured. After 5 mins, 2-phenyl4,4,5,5-tetramethylimidazoline-1-oxyl 3-oxide, PTIO, (1 mM) was applied (FIG. 12). Then after 11 mins, Ap₂CH₂P₂A (2.5 μM) was applied.

The use of PTIO reduced the extent of AppCH₂ppA-mediated inhibition of orthodromically evoked field potentials by over 50% (FIG. 12).

Nitric oxide (NO) has been shown to mediate adenosine outflow in response to P2-receptor activation.²⁰ Thus, the use of PTIO, a known NO specific scavenger, would be expected to affect the AppCH₂ppA-mediated inhibition of orthodromically evoked field potentials if this involves a P2 receptor. The observed reduction is consistent with a P2 receptor having a direct role in this case. Thus, it may be postulated that AppCH₂ppA-mediated effects proceed by a pathway that links PPADS-sensitive P2 receptor activation, resulting from the binding of AppCH₂ppA, with the production of NO that subsequently stimulates the intracellular synthesis of adenosine leading to exclusive postsynaptic A1 receptor activation.

### Experiment 12

The time course of the changes of amplitude in orthodromically induced population spikes in CA1 zone of rat hippocampal slices prepared in accordance with the general procedure was measured. After 10 mins, adenosine deaminase (approx. 2 U/ml) was applied. Then after 20 mins, Ap₂CH₂p₂A (7.4 μM) was applied.

The AppCH₂ppA induced inhibition of orthodromically evoked field potentials was eliminated by the presence of adenosine deaminase.

Nucleoside-activated receptors have been observed to bring about presynaptic inhibition of glutamate release in hippocampal neurons in an earlier study.²¹ This process is mediated by so-called P3 receptors (P2Y-theophylline-sensitive receptors) and has some similarities to the observed AppCH₂ppA effects. However, the fact that AppCH₂ppA effects were eliminated by adenosine deaminase is inconsistent with a P3 mechanism, indicating that AppCH₂ppA effects are not mediated through a similar pathway.

### Experiment 13

The time course of the changes of amplitude in orthodromically induced population spikes in CA1 zone of rat hippocampal slices prepared in accordance with the general procedure was measured before and after the addition of Ap₂CH₂p₂A (2.5 μM) at 22° C. (FIG. 13) and at 36° C. (FIG. 14).

The temperature dependence of this effect is consistent with a signalling pathway that involves the diffusion of small molecule mediators such as adenosine.

### Experiment 14

Modulation of NMDA-receptor mediated current by non-hydrolysible analogues of diadenosine polyphosphate.

Diadenosine polyphosphates are natural compounds that can play a neurotransmitter role in the synaptic terminals of the central nervous system. Here we demonstrate that non-hydrolysible analogue of diadenosine polyphosphates AppCH₂ppA may affect the functioning of NMDA-receptor-mediated channels. In isolated hippocampal pyramidal neurons, AppCH2ppA applied at low micromolar concentrations increased the amplitude of the NMDA-activated current in a concentration-dependent manner. These effects of AppCH₂ppA were eliminated in the presence of purine P2 receptors antagonists PPADS and reactive blue, suggesting that effects of AppCH₂ppA are mediated by activation of purine receptors. The effects of AppCH₂ppA were removed in the presence EDTA, indicating that some of bivalent cations, tonically present in extracellular solution in the trace amount are involved in the observed effects downstreams of the activation of P2-receptors. Furthermore the effects of AppCH₂ppA were abolished after pre-treatment of neurons with the non-specific inhibitor of tyrosine protein kinase inhibitor genestein. These data taken together allow suggesting that AppCH₂ppA potentiated NMDA-currents is due to P2 receptor-dependent activation of tyrosine kinase via reducing the tonic inhibition of NMDA receptors by some of bivalent cations, most probably Zn²⁺.

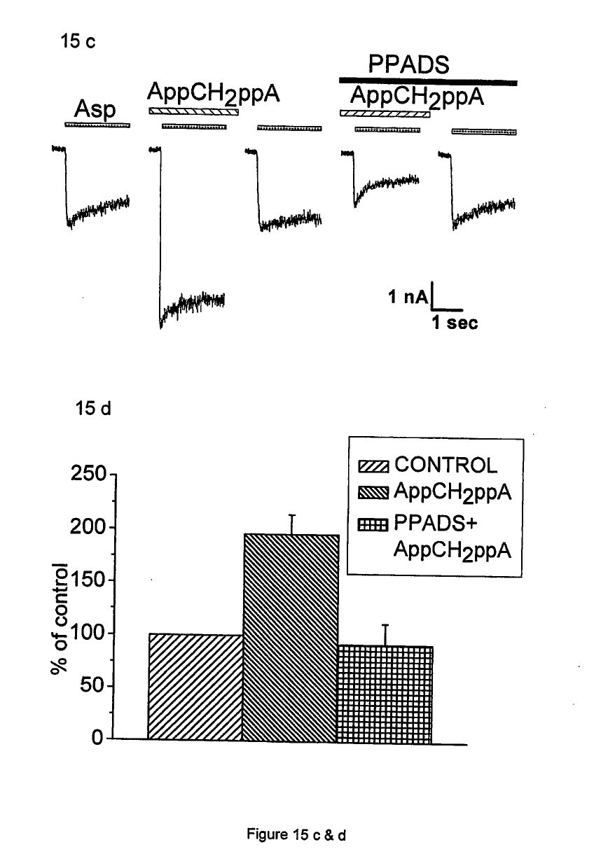

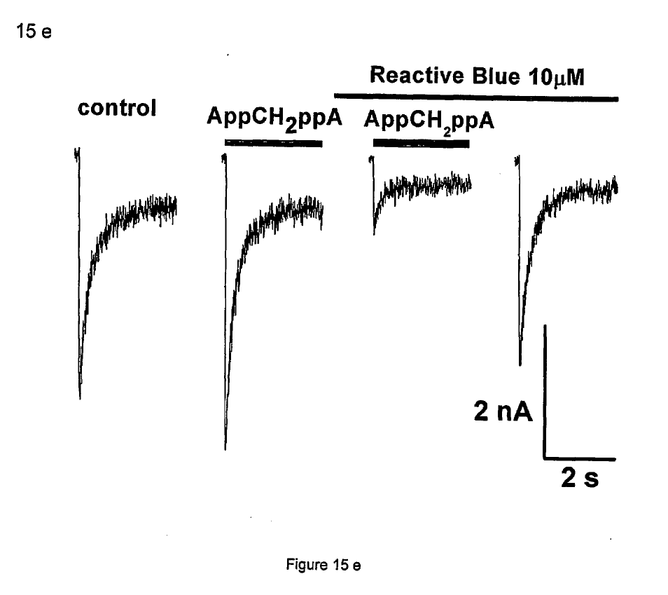

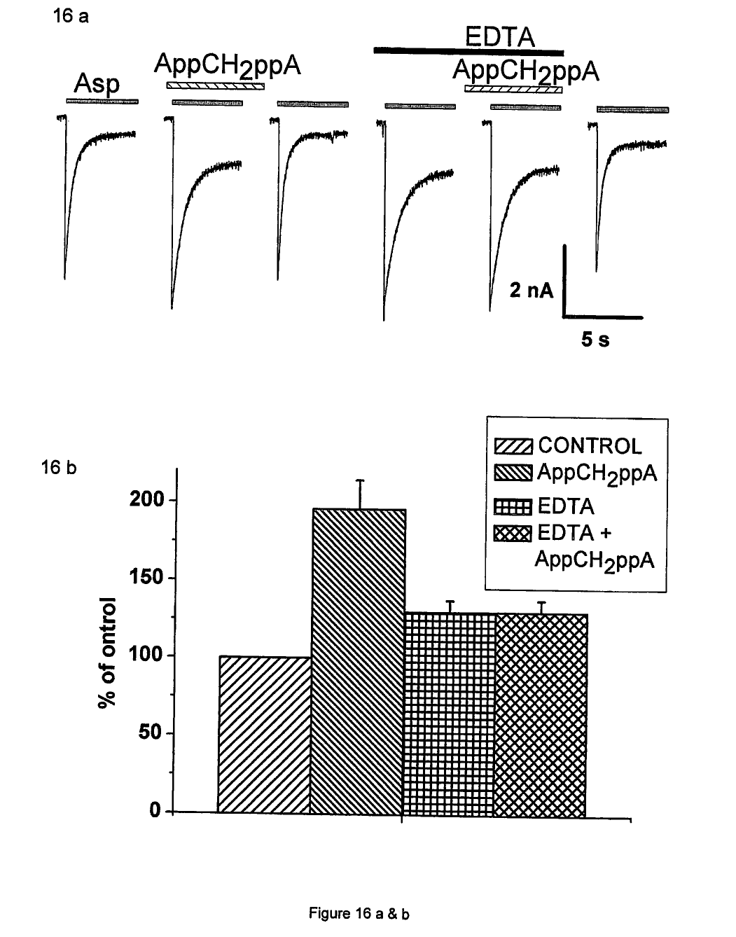

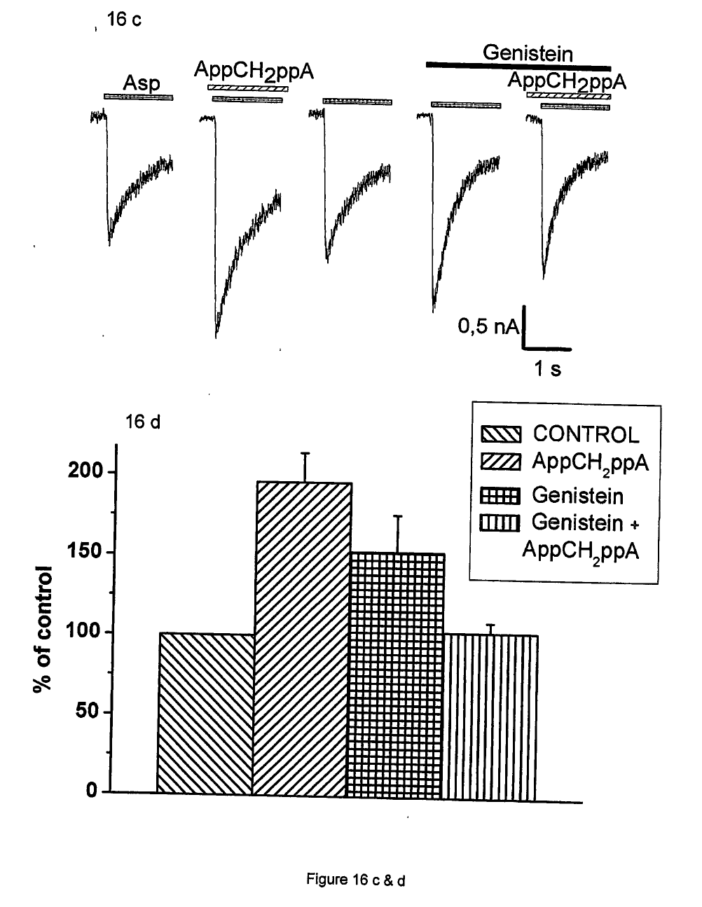

The results are shown in FIGS. 15 and 16.

FIG. 15 shows a modulation of NMDA receptor-activated currents recorded in isolated hippocampal pyramidal neurons by AppCH2ppA (1 μM) is mediated by purine P2 receptors.

NMDA-receptor-activated currents were evoked by 1-2 sec long co-application of aspartate (ASP) (1 mM) and glycine (10 μM). Vh=−100 mV in Mg²⁺ free solution.

(a) Representative traces of NMDA-activated current in control, in the presence and after wash-out of AppCH₂ppA.

(b) Statistics for the effects of AppCH₂ppA and other purine agonists; ADP(1 μM), UTP(1 μM), UDP (1 μM) on the peak amplitude of NMDA-current,

(c) Inhibition of AppCH₂ppA—mediated enhancement of NMDA-currents by non-specific purine P2 receptor antagonist PPADS.

(d) statistics for the effects of AppCH₂ppA on the peak amplitude of NMDA-current in control conditions and in the presence of P2 antagonists PPADS.

(e) Inhibition of AppCH₂ppA—mediated effects by P2Y receptor antagonists Reactive blue (RB).

FIG. 16 shows a modulation of NMDA receptor-activated currents by AppCH₂ppA is mediated by relief from tonic inhibition by bivalent cations.

(a) Potentiation of NMDA—activated currents is eliminated in the presence of chelator of bivalent cations EDTA. Representative traces of NMDA-activated current in control, in the presence of EDTA and in the presence of AppCH₂ppA and EDTA.

(b) Statistics for the effects of AppCH₂ppA on the peak amplitude of NMDA-current in control conditions and after pre-application of EDTA.

(c) Inhibition of AppCH₂ppA—mediated enhancement of NMDA-currents by non-specific inhibitor of tyrosine protein kinase genestein.

(d) statistics for the effects of AppCH₂ppA on the peak amplitude of NMDA-current in control conditions and after pre-treatment of genestein.

### MATERIALS AND METHODS

### Materials

All the chemicals for intra- and extra-cellular solutions were purchased from Sigma Chemical Co. (St. Louis, Mo., USA).

### Cell Preparation

Wistar rats (12-17-days old) were decapitated under ether anaesthesia and the hippocampus (or cerebellum) was removed. It was cut into slices (300-500 μm) in a solution containing (in mM): 150 NaCl; 5 KCl; 1.25 NaH₂PO₄; 26 NaHCO₃; 1.1 MgCl₂; 10 glucose; pH 7.4. Then the slices were incubated for 10 min at 32° C. with 0.5 mg/ml of protease (type XXIII) from Aspergillus oryzae. Single pyramidal cells from CA1 and CA3 stratum pyramidale layers were isolated by vibrodissociation locally in the stratum pyramidale CA3 and CA1 hippocampal pyramidal neurons were identified by their characteristic form and partially preserved dendritic arborisation.

After isolation the cells were usually suitable for recordings for 2-4 h. Throughout the entire procedure the solutions with the slices were continuously saturated with 95% O₂ and 5% CO₂ gas mixture to maintain pH 7.4. The tested substances were dissolved in DMSO to a stock concentration of 10 mM and kept frozen at −40° C. in daily aliquots. The substances were dissolved in external saline to their final concentration immediately before the experiments.

### Current Recordings

NMDA-activated currents in isolated neurons were induced by the step application of aspartate (1 mM) and glycine (1 mM) in the “concentration clamp” mode (Krishtal et al., 1983), using the computerized “Pharma-Robot” set-up (Pharma-Robot, Kiev). This equipment allows a complete change of saline within 15 ms. Transmembrane currents were recorded using a conventional patch-clamp technique, in the whole-cell configuration. Patch-clamp electrodes were pulled with a horizontal puller (Sutter Instruments) and had an internal tip diameter between 1.4 and 1.8 μm and a tip resistance between 2.5 and 5 MOm. The intracellular solution contained (in mM): 70 Tris-PO₄; 5 EGTA; 40 TEA-Cl (tetraethylammonium chloride); 30 Tris-Cl; 5 Mg-ATP; 0.5 GTP; pH 7.2. The composition of extracellular solution was (in mM):. 130 NaCl; 3 CaCl₂; 5 KCl; 2 MgCl₂; 10 HEPES-NaOH; 0.1 μM TTX; pH 7.4. Recording of the currents was performed using patch-clamp amplifiers (DAGAN, USA). Transmembrane currents were filtered at 3 kHz, stored and analysed with an IBM-PC computer using homemade software. NMDA responses were recorded with a 3 min interval. All experiments were performed at room temperature (19-24° C.).

### Experiment 15

### Antinociceptive Activity of AppCH₂ppA

Experimental Procedure:

CFA-Induced Thermal Hyperalgesia. Unilateral inflammation was induced by injecting 100 μl of 50% solution of CFA (Sigma) in physiological saline into the plantar surface of the right hind paw of the rat. The hyperalgesia to thermal stimulation was determined 48 h after CFA injections using the same apparatus as described below for the noxious acute thermal assay.

Thermal sensitivity: On each day following mechanical testing, rats will be placed in a thermal testing apparatus (Plantar test, Ugo Basile, Italy), in which they will be free to move. After 30 min of acclimatization, a constant-power IR stimulus will be focused through the glass base on to the sole of the foot, and the latency for reflex foot withdrawal will be recorded automatically via a photoelectric monitor as previously described Hargreaves et al. 1988.

In each test session, each rat (from 6 tested rats) was tested in three sequential trials at approximately 15 min intervals.

### Results

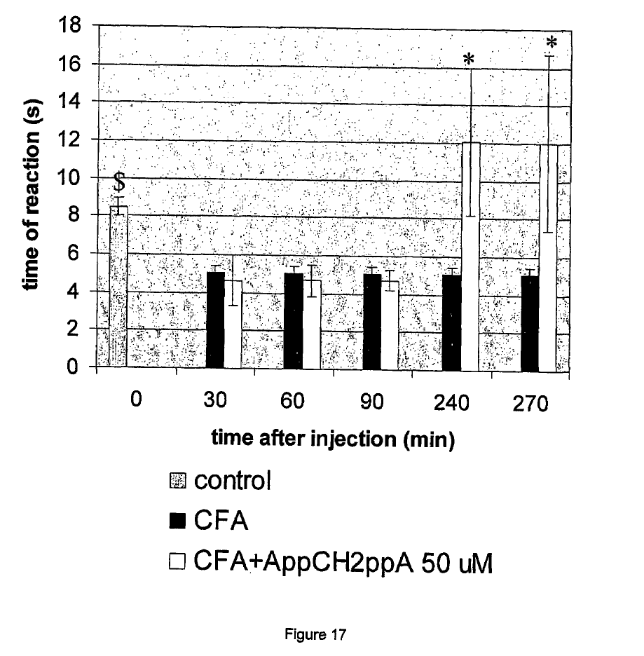

Antinociceptive activity of AppCH₂ppA. To characterize the nociceptive activity of AppCH₂ppA the effects of this compound was evaluated in CFA-induced thermal hyperalgesia animal model after s.c. administration. AppCH₂ppA was evidently potent in reducing thermal hyperalgesia (FIG. 17).

After 48 h of inflammation induced by the intraplantar administration of CFA, AppCH₂ppA fully blocked thermal hyperalgesia (FIG. 17). The antinociceptive effects of AppCH₂ppA were specific to the injured paw, as the paw withdrawal latencies for the uninjured paw were less effectively altered by AppCH₂ppA at the doses tested. The antinociceptive effects of AppCH₂ppA in the injured paw were delayed in onset and appeared after 3 hours after injection.

FIG. 17—AppCH₂ppA increases paw withdrawal latencies 48 h after intraplantar administration of CFA. Responses (paw withdrawal latencies (mean±SEM)) in control and CFA-injected paw.

AppCH₂ppA (50 μmol/kg s.c.), attenuates CFA-induced thermal hyperalgesia in the rat

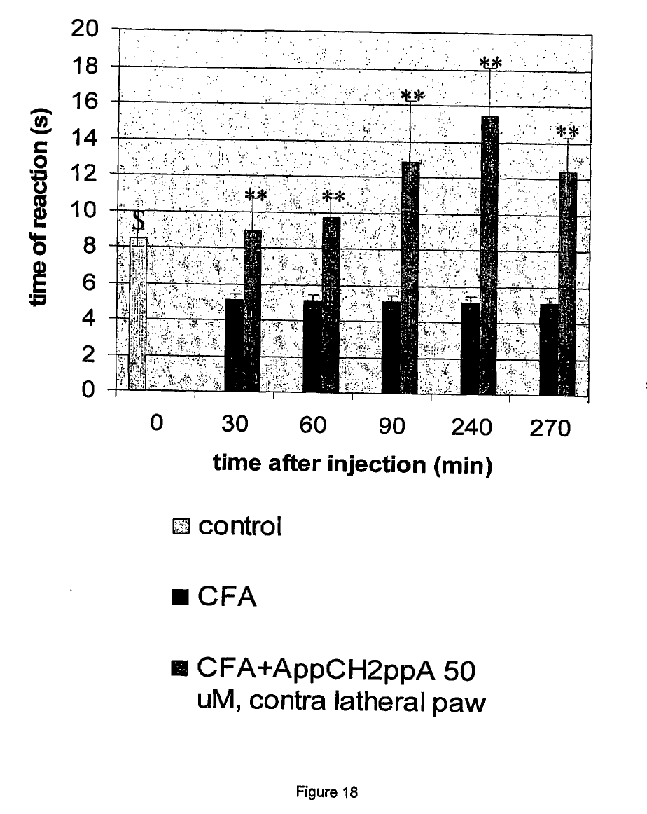

FIG. 18—AppCH₂ppA increases paw withdrawal latencies 48 h in contra lateral (non-inflamed) paw. Responses (paw withdrawal latencies (mean±SEM)) in control and contra lateral paw.

AppCH₂ppA (50 μmol/kg s.c.), attenuates thermal hyperalgesia in the rat.

* P<0.05 CFA vs CFA+AP4 50 uM

$ P<0.05 CFA vs control

** P<0.05 CFA vs contra latheral paw CFA+AppCH2ppA 50 uM

P control vs CFA 1.58E-12

P CFA vs CFA+AP4 5 uM 0.000314

P CFA vs CFA+AP4 50 uM 0.000501

P CFA vs contra latheral paw CFA+AP4 50 uM 0.0043

P CFA vs contra latheral paw CFA+AP4 50 uM 0.000335

P CFA vs contra latheral paw CFA+AP4 50 uM 3.67E-06

P CFA vs contra latheral paw CFA+AP4 50 uM 1.53E-07

P CFA vs contra latheral paw CFA+AP4 50 uM 6.54E-05

### Apparatus Description

7370-Plantar Test

### Measurement of Hyperalgesia to Thermal Stimulation in Unrestrained Animals

Featuring:

Automatic detection of the behavioral end point

Validity unaffected by repeated testing

Greater bioassay sensitivity than other thermal or mechanical tests

Each animal can serve as its own control

The Instrument basically consists of:

a Movable I.R. (infra-red) Source

a Glass Pane onto which the Rat Enclosure is located

a Controller

A 3-compartment enclosure has been provided to speed up the test when a number of animals is involved. In each compartment the animal is unrestrained.

After the acclimation period, the I.R. Source placed under the glass floor (see the picture) is positioned by the operator directly beneath the hind paw. A trial is commenced by depressing a key which turns on the I.R. Source and starts a digital solid state timer.

When the rat feels pain and withdraws its paw, the sudden drop of reflected radiation switches off the I.R. Source and stops the reaction time counter.

The withdrawal latency to the nearest 0.1 s is determined.

### Calibration Radiometer

Each Plantar Test is accurately calibrated via an I.R. Radiometer to make sure that its I.R. source delivers the same power flux (expressed in mW per square cm) and hence a nociceptive stimulus of the same intensity.

The end user should consider this extremely useful accessory, the Heat-Flow I.R. Radiometer Cat 37300, a battery operated, self sufficient instrument complete with I.R. probe, digital meter and adaptors for the Tail Flick and Plantar Test, all parts neatly lodged in a sturdy plastic case with punched foam lining.

The 37300 Radiometer enables the experimenter to:

i) Check (and adjust if necessary) the I.R. emission. In fact, the I.R. output of the Plantar Test may in the course of one-two years undergo to 2-3% reduction, due to dust gathered on the optics, blackening of the I.R. bulb, accidental knocks, ageing of components due to thermal cycles, etc.

Moreover, in case the bulb is replaced or the electronics serviced, output alteration of more significant magnitude, say, 8-10%, may take place.

ii) Make sure that two or more Plantar-Test units deliver thermal nociceptive stimuli of exactly the same intensity. Balance them, if necessary.

iii) Know the I.R. energy (1 mW for the duration of is corresponds to 1 mJ) in absolute terms, a useful datum to compare with any equal or different method/instrument described in the literature.

### BIBLIOGRAPHY

Method Paper:

K. M. Hargreaves, R. Dubner, F. Brown, C. Flores and J. Joris: “A New and Sensitive 5 Method for Measuring Thermal Nociception in Cutaneous Hyperalgesia.” Pain 32: 77-88, 1988.

Additional Papers:

K. M. Hargreaves, R. Dubner and J. Joris: “Peripheral Action of Opiates in the Blockade of Carrageenan-Induced Inflammation” Pain Research and Clinical Management. Vol. 3. Elsevier Science Publishers, Amsterdam: 55-60, 1988

G. Benneth and Y. K. Xie: “A Peripheral Neuropathy in Rat that Produces Disorders of Pain Sensation Like Those Seen in Man” Pain 33: 87-107, 1988.

M. Iadarola and G. Draisci: “Elevation of Spinal Cord Dynorphin mRNA Compared to Dorsal Root Ganglion Peptide mRNAs During Peripheral Inflammation” In: The Arthritic Rat as a Model of Clinical Pain? by J. Besson and G. Guilbaud (eds.) Elsevier Press, Amsterdam: 173-183, 1988.

A. Costello and K. M. Hargreaves: “Suppression of Carrageenan-Induced Hyperalgesia. Edema and Hyperthermia by a Bradykinin Antagonist” European J. Pharmacol., 1989.

K. M. Hargreaves, R. Dubner and A. Costello: “Corticotropin Releasing Factor (CRF) has a Peripheral Site of Action for Antinociception” European J. Pharmacol., 1989.

J. Hylden, R. Nahin, R. Traub and R. Dubner: “Expansion of Receptive Fields of Spinal Lamina I Protection Neurons in Rats with Unilateral Adjuvant-induced Inflamma-tion: The Contribution of Central Dorsal Horn Mechanisms” Pain 37: 229-244, 1989.

In addition, more than 30 abstracts using this device have been presented at U.S. (eg. Society for Neuroscience) and International (e.g., International Association for the Study of Pain) scientific meeting

http://www.ugobasile.com/site/manuals/condensed_catalogue.pdf

http://www.ugobasile.com/site/product2.asp?ID=3

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims

### REFERENCES

1. Pintor, J.; Gualix, J.; Miras-Portugal, M. T. Mol. Pharm., 1997, 51, 277-284.

2. Oaknin, S.; Rodriguez-Ferrer, C. R.; Aguilar, J. S.; Ramos, A.; Rotlian, P. Neurosci. Lett., 2001, 309, 177-180.

3. Ap₄A and other dinucleoside polyphosphates, Ed. A. G. McLennan, CRC Press, Boca Raton, Fla., 1992.

4. Plateau, P.; Blanquet S., Adv. Micro. Physiol., 1994, 36, 81.

5. Wang, Y., Chang, C. F., Morales, M., Chiang, Y. H., Harvey, B. K., Su, T. P., Tsao, L. I., Chen, S., Thiemermann, C. J. Neuroscience 2003, 23, 7958-65.

6. Ortiz, B.; Sillero, A.; Gunther Sillero, M. A.; Eur. J. Biochem., 1993, 212, 263.

7. Theoclitou, M.-E.; Wittung, E. P. L.; Hindley, A. D.; El-Thaher, T. S. H.; Miller, A. D. J. Chem. Soc., Perkin Trans. 1, 1996, 2009-2019.

8. McLennan, A. G. Pharmacol Ther, 2000, 87, 73-89.

9. Wright, M., Tanner, J. A., and Miller, A. D. Anal Biochem, 2003, 316, 135-138.

10. Guranowski, A. Pharmacol Ther, 2000, 87, 117-39.

11. Theoclitou, M. E.; El-Thaher, T. S. H.; Miller, A. D. J. Chem. Soc., Chem. Commun., 1994, 659-661.

12. Wright, M.; Miller, A. D. Bioorg. Med. Chem. Lett., 2004, 14, 2813-2816.

13. Ng, K. E.; Orgel, L. E. Nucleic. Acids. Res., 1987, 15 (8), 3573-3580.

14. Klishin, A.; Lozovaya, N.; Pintor, J.; Miras-Portugal, M. T.; Krishtal, O. Neuroscience, 1994, 58, 235-6.

15. Pintor, J.; Diaz-Hernandez, M.; Gualix, J.; Gomez-Villafuertes, R.; Hernando, F.; Miras-Portugal, M. T. Pharmacol Ther, 2000, 87, 103-15.

16. Bianchi, B. R.; Lynch, K. J.; Touma, E.; Niforatos, W.; Burgard, E. C.; Alexander, K. M.; Park, H. S.; Yu, H.; Metzger, R.; Kowaluk, E.; Jarvis, M. F.; van Biesen, T. Eur J Pharmacol, 1999, 376, 127-38.

17. Pintor, J.; Miras-Portugal, M. T. Br J Pharmacol, 1995, 115, 895-902.

18. Greene, R. W.; Haas, H. L. Prog Neurobiology, 1991, 36, 329-41.

19. Klishin, A.; Lozovaya, N.; Krishtal, O. Neuroscience, 1995, 65, 947-53.

20. Juranyi, Z.; Sperlagh, B.; Vizi, E. S. Brain Res, 1999, 823, 183-90.

21. Mendoza-Fernandez, V.; Andrew, R. D.; Barajas-Lopez, C. 2000 J Pharmacol Exp Ther, 2000, 293, 172-9.

22. Rudolphi K; Schubert P; Parkinson F. E.; Fredholm B. B. Trends Pharmacol Sci. 1992, 13, 439-445.

23. Knutsen L. J. S.; Murray T. F. Adenosine and ATP in epilepsy. In Purinergic Approaches in Experimental Therapeutics. Eds. Jacobson K. A,; Jarvis M. F. Wiley-Liss, New York, 1997, pp. 423-447.

24. Dragunow M. Adenosine and Adenine Nucleotides as Regulators of Cellular Function. Ed. Phillis J. W., Boca Raton, Fla., CRC Press, 1991, 367-379.

25. IJzerman A. P.; van der Wenden N. M. Purinergic Approaches in Experimental Therapeutics Ed. Jacobson, K. A.; Jarvis, M. F., Wiley-Liss, Inc. New York, 1997, 129-148.

26. Firestein G. Drug Dev. Res. 1996; 39: 371-376.

27. Ferre S.; Fredholm B. B.; Morelli M.; Popoli P.; Fuxe K. Trends Neurosci 1997; 20:482-487.

28. Carley D.; Radulovacki M.; Purinergic Approaches in experimental Therapeutics. Eds. Jacobson K. A.; Jarvis M. F., Wiley-Liss, New York, pp.515-526.

29. Sawynok J. Eur J Pharmacol 1998; 347:1-11.

30. Cook S. P.; Vulchanova L.; Hargreaves K. M.; Elde R.; McCleskey E. W. Nature 1997; 387:505-508.

31. Burnstock G. A. Lancet 1996, 347, 1604-1605.

32. Vulchanova L.; Arvidsson U.; Riedl M.; Wang J.; Buell G.; Surprenant A.; North R. A.; Elde R.; Proc. Natl. Acad. Sci. U.S.A. 1996, 93, 8063-8067.

33. Keil G. J.; DeLander G. E. Life Sci 1992; 51:L171-L176.

34 Kowaluk E. A.; Bhagwat S. S.; Jarvis M. F. Curr. Pharmaceut. Design 1998; 4: 403-416.

35. Poon A.; Sawynok J. Pain 1998; 74:235-245.

36. Lee Y. W.; Yaksh T. L. J Pharmacol Exp Ther 1996; 277: 1642-1648.

37. Segerdahl M.; Ekblom A.; Sandelin K.; Wickman M.; Sollevi A. Anesth Analg 1995; 80:1145-1149.

38. Sollevi A. Acta Anaesthesiol Scand Suppl 1997; 110:135-136.

39. Woolf, C. J. Textbook of Pain, 3rd Ed. Eds Wall, P.; Melzack, R. D., Churchill Livingstone, Edinburgh, 1994, 101-112.

40. Fukunaga A. F. Purinergic Approaches in Experimental Therapeutics. Eds Jacobson K. A., Jarvis M. F., Wiley-Liss, New York, 1997, pp. 471-493.

1. Use of a compound of formula (1): 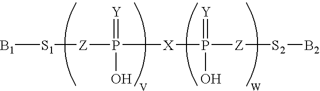
or a pharmaceutically acceptable salt thereof,
in the manufacture of a medicament for use in one or more of:
treatment of ischemia,
as a neurological protection agent;
as a tissue protection agent;
treatment of pain; and
treatment of inflammation;
wherein X, is selected from 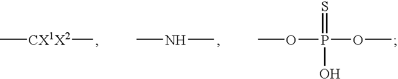
wherein X¹ and X² are independently selected from H, Cl, Br and F;
each Y is independently selected from S and O;
each Z is independently selected from
—CX³X⁴—, —NH—, —O—;
wherein X3 and X4 are selected from H, Cl, Br and F;
B₁ and B₂ are independently selected from adenine, guanine, xanthine, thymine, uracil, cytosine and inosine;
S₁ and S₂ are independently selected from ribose, 2′-deoxyribose, 3′deoxyribose, arabinofuranoside and ring opened forms thereof.
V is selected from 0, 1, 2, 3, 4 and 5;
W is selected from 0, 1, 2, 3, 4 and 5; and
V plus W is an integer from 2 to 6.

2. Use of a compound of formula (1) according to claim 1 wherein at least one of B₁ and B₂ is adenine.

3. Use of a compound of formula (1) according to claim 1 wherein B₁ and B₂ are both adenine.

4. Use of a compound of formula (1) according to claim 1 wherein S₁ and S₂ are the same.

5. Use of a compound of formula (1) according to claim 4 wherein S₁ and S₂ are ribose.

6. Use of a compound of formula (1) according to claim 1 wherein each Z is 0.

7. Use of a compound of formula (1) according to claim 1 wherein V is 2.

8. Use of a compound of formula (1) according to claim 1 wherein W is 2.

9. Use of a compound of formula (1) according to claim 1 wherein X is
—CX¹X²—.

10. Use of a compound of formula (1) according to claim 1 wherein and X¹ and X² are both H.

11. Use of compound of formula (1) according to claim 1, or a pharmaceutically acceptable salt thereof in the manufacture of a medicament for use in one or more of: (a) treatment of diseases and medical conditions associated with P2-receptors;
(b) treatment of diseases and medical conditions associated with Al adenosine receptors;
(c) moderating the activity of P2-receptors;
(d) moderating the activity of Al adenosine receptors; and
(e) for modulating K+ influx via G protein-gated inwardly rectifying K⁺ (GIRK) channels in mammalian cells.

12. A compound selected from:
(a) AppₛpA 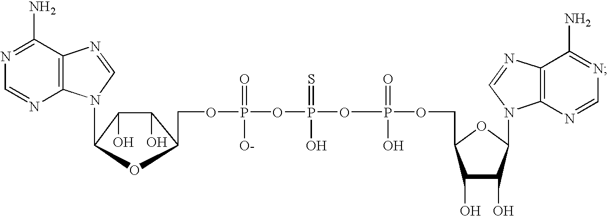
(b) A_{diol}ppCH2ppA_{diol} 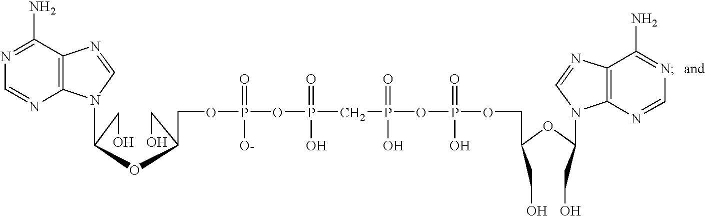
(c) AppNHpppU

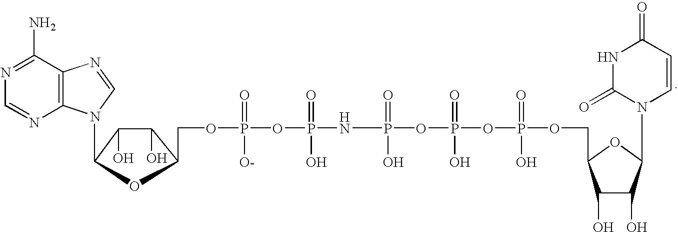

13. (canceled)
